(19) United States
(12) Patent Application Publication
(10) Pub. No.: US 2018/0325380 A1
(43) Pub. Date: Nov. 15, 2018

(54) SUBJECT INFORMATION ACQUISITION DEVICE AND SUBJECT INFORMATION ACQUISITION METHOD

(71) Applicant: CANON KABUSHIKI KAISHA, Tokyo (JP)

(72) Inventor: NAOTO ABE, Machida-shi (JP)

(21) Appl. No.: 15/973,929

(22) Filed: May 8, 2018

(30) Foreign Application Priority Data
May 15, 2017 (JP) 2017-096747

(51) Int. Cl. A61B 5/00

(52) U.S. Cl. CPC A61B 5/0095

(57) ABSTRACT

A subject information acquisition device having a light source for emitting light to a subject; an acoustic wave detection unit configured to receive an acoustic wave generated by the subject in response to the light, and convert the received acoustic wave into an electric signal; a signal processing unit configured to implement emission of the light and acquisition of the electric signal at a non-periodic sampling timing, and add together electric signals acquired in time series at each sampling timing; and an image generation unit configured to generate an image representing characteristic information of the subject on the basis of the added electric signals.

### BACKGROUND OF THE INVENTION

### Field of the Invention

The present invention relates to a subject information acquisition device that uses a photoacoustic effect.

### Description of the Related Art

In medical fields, recent years have witnessed advances in research into techniques for imaging structural information and physiological information, or in other words functional information, relating to the interior of a subject. Photoacoustic tomography (PAT) has recently been proposed as one of these techniques.

When a living organism serving as a subject is irradiated with light such as a laser beam, an acoustic wave (typically an ultrasonic wave) is generated as the light is absorbed by biological tissue in the subject. This phenomenon is known as the photoacoustic effect, and an acoustic wave generated by the photoacoustic effect is known as a photoacoustic wave. Tissues constituting the subject absorb optical energy at different absorption rates, leading to corresponding variation in the acoustic pressure of the generated photoacoustic wave. In PAT, characteristic information relating to the interior of the subject can be acquired by receiving generated photoacoustic waves using a probe, and mathematically analyzing reception signals.

In the field of photoacoustic devices, as in the field of ultrasonic diagnosis devices, research and development have been undertaken in relation to a device with which an observation site can be accessed easily using a handheld probe. Further, in the field of photoacoustic devices shaped as handheld probes, research and development have been undertaken in relation to a device with which a structural image or a functional image of the interior of a subject can be observed in real time.

A photoacoustic device reconstructs an image on the basis of weak acoustic waves generated in the interior of the subject, and therefore numerous means have been proposed for improving an S/N ratio. For example, Japanese Patent Application Publication No. 2016-47102 discloses a photoacoustic device that emits light to a subject a plurality of times, receives acoustic waves, and averages the plurality of acquired signals. By generating a photoacoustic image on the basis of averaged signals, noise can be reduced, leading to an improvement in image quality.

In the photoacoustic device described in Japanese Patent Application Publication No. 2016-47102, signals acquired at each sampling period are averaged, and therefore randomly intermixed noise can be suppressed. With this device, however, a sufficient suppression effect cannot be acquired in relation to periodically generated noise.

### SUMMARY OF THE INVENTION

An object of the present invention is to suppress noise generated periodically in a photoacoustic device which may have occurred in the prior art.

An aspect of the invention is a subject information acquisition device comprising: a light source for emitting light to a subject; an acoustic wave detection unit configured to receive an acoustic wave generated by the subject in response to the light, and convert the received acoustic wave into an electric signal; a signal processing unit configured to implement emission of the light and acquisition of the electric signal at a non-periodic sampling timing, and add together electric signals acquired in time series at each sampling timing; and an image generation unit configured to generate an image representing characteristic information of the subject on the basis of the added electric signals.

Another aspect of the invention is a subject information acquisition method comprising: an emission step for emitting light; an acoustic wave detection step for receiving an acoustic wave generated by the subject in response to the light, and converting the received acoustic wave into an electric signal; a signal processing step for implementing emission of the light and acquisition of the electric signal at a non-periodic sampling timing, and adding together electric signals acquired in time series at each sampling timing; and an image generation step for generating an image representing characteristic information of the subject on the basis of the added electric signals.

According to the present invention, noise generated periodically in a photoacoustic device can be suppressed.

Further features of the present invention will become apparent from the following description of exemplary embodiments with reference to the attached drawings.

### BRIEF DESCRIPTION OF THE DRAWINGS

FIG. 1 is a functional block diagram of a photoacoustic device according to a first embodiment;

FIG. 2A is a schematic view of a handheld probe according to the first embodiment;

FIG. 2B is a schematic view of the handheld probe according to the first embodiment;

FIG. 3 is a diagram showing configurations of a computer and peripheral devices according to the first embodiment;

FIG. 4A is a diagram illustrating operation timings according to the first embodiment;

FIG. 4B is a diagram illustrating operation timings according to the first embodiment;

FIG. 4C is a diagram illustrating operation timings according to the first embodiment;

FIG. 5 is a diagram illustrating operation timings according to a second embodiment;

FIG. 6A is a diagram illustrating a problem to be solved by the present invention; and

FIG. 6B is a diagram illustrating a method for solving the problem.

### DESCRIPTION OF THE EMBODIMENTS

Preferred embodiments of the present invention will be described below with reference to the figures. Note, however, that dimensions, materials, shapes, relative arrangements, and so on of constituent components described below may be modified as appropriate in accordance with the configuration of the device to which the invention is applied and various conditions. Accordingly, the scope of the present invention is not limited to the following description.

The present invention relates to a technique for detecting acoustic waves propagating from a subject in order to generate and acquire characteristic information relating to the interior of the subject. The present invention may therefore be taken as a subject information acquisition device and a control method therefor, or a subject information acquisition method. The present invention may also be taken as a program for causing an information processing device having hardware resources such as a CPU and a memory to execute these methods, and a non-temporary storage medium for storing the program so that the program can be read by a computer.

The subject information acquisition device according to the present invention uses the photoacoustic effect to receive acoustic waves generated in the interior of a subject by emitting light (an electromagnetic wave) to the subject, and acquire characteristic information relating to the subject in the form of image data. In this case, the characteristic information is information indicating characteristic values corresponding respectively to a plurality of positions within the subject, and is generated using reception signals acquired by receiving photoacoustic waves.

The characteristic information acquired by photoacoustic measurement is a value reflecting an optical energy absorption rate. For example, the characteristic information includes a generation source of an acoustic wave generated in response to light emission, an initial acoustic pressure within the subject or an optical energy absorption density and an optical energy absorption coefficient derived from the initial acoustic pressure, and a concentration of a tissue-forming substance.

Further, by determining an oxyhemoglobin concentration and a deoxyhemoglobin concentration as the substance concentration, an oxygen saturation distribution can be calculated. A glucose concentration, a collagen concentration, a melanin concentration, fat and water volume fractions, and so on can also be determined. Moreover, substances having a distinguishing light absorption spectrum, for example a contrast medium such as indocyanine green (ICG) delivered into the body, may be used as subjects.

On the basis of the characteristic information relating to respective positions within the subject, a two-dimensional or three-dimensional characteristic information distribution is acquired. Distribution data can be generated in the form of image data. The characteristic information may be determined as distribution information relating to respective positions within the subject, rather than as numerical value data. More specifically, the distribution information may indicate an initial acoustic pressure distribution, an energy absorption density distribution, an absorption coefficient distribution, an oxygen saturation distribution, and so on.

The acoustic wave according to this specification is typically an ultrasonic wave, but includes elastic waves referred to as sound waves and acoustic waves. An electric signal converted from an acoustic wave using a probe or the like will also be referred to as an acoustic signal. Note, however, that the terms ultrasonic wave and acoustic wave as used in this specification are not intended to limit the wavelength of the corresponding elastic waves. An acoustic wave generated by the photoacoustic effect will be referred to as a photoacoustic wave or an optical ultrasonic wave. An electric signal derived from a photoacoustic wave will also be referred to as a photoacoustic signal. Note that in this specification, a photoacoustic signal includes both an analog signal and a digital signal. The distribution data will also be referred to as photoacoustic image data and reconstructed image data.

### First Embodiment

<Outline of device>

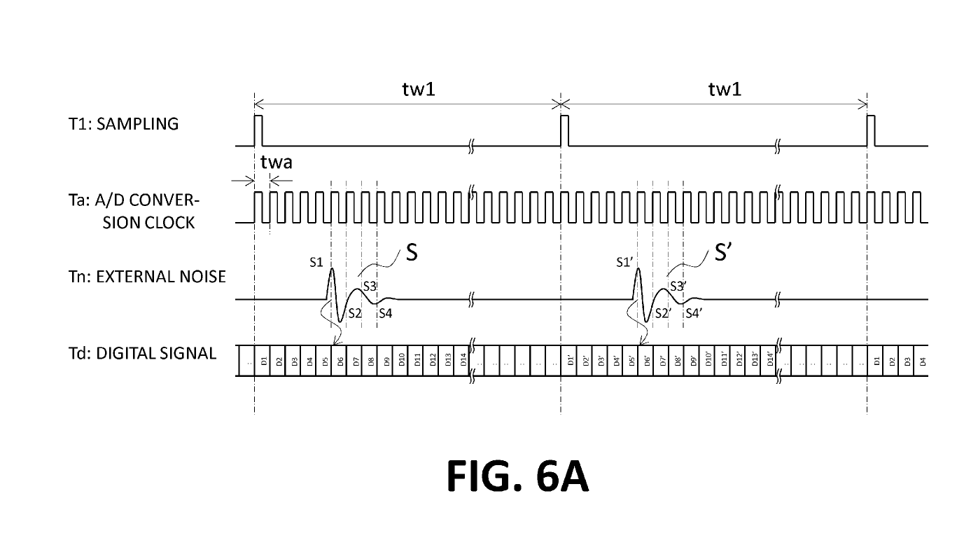

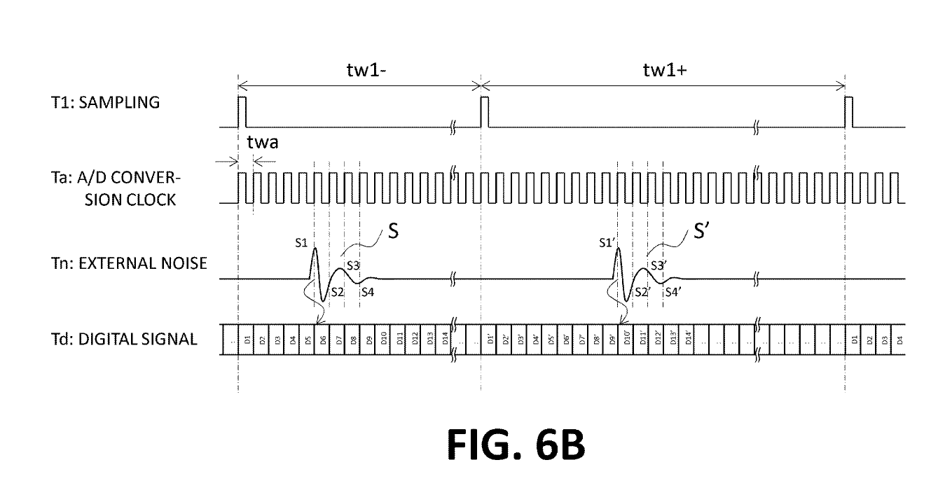

A problem to be solved by the present invention will now be described with reference to FIGS. 6A and 6B.

FIG. 6A is a timing diagram illustrating a problem occurring in the prior art. In FIG. 6A, the horizontal axis is a temporal axis.

First, effects of external noise on a photoacoustic device will be described. In FIG. 6A, T1 denotes a clock used for data sampling. Here, a sampling interval (a first period according to the present invention) is set at tw1. In this example, a light source of the photoacoustic device emits light at a rising edge of the sampling clock T1, whereupon a photoacoustic signal generated in response to emission of the light is acquired as time series data in each sampling period. Here, in a photoacoustic device according to the prior art, the sampling period tw1 is fixed, or in other words sampling is executed periodically.

Ta in FIG. 6A denotes an A/D conversion clock. In the photoacoustic device, an A/D converter converts a photoacoustic signal constituted by an analog signal into a digital signal at a rising edge of the A/D conversion clock. Then, as indicated by Td, digital signals (D1, D2, D3, . . . ) are acquired in time series using the light emission timing of the light source as a reference.

However, the photoacoustic waves generated in the interior of the subject when the subject is irradiated with light (in particular, light from a semiconductor light emitting element rather than a laser light source) are extremely weak. In a typical photoacoustic device, therefore, electric signals (digital signals) acquired in time series at each fixed period are averaged in order to improve the S/N ratio of the photoacoustic signal. Note that in order to improve the S/N ratio of the photoacoustic signal, a large number of signals must be averaged, but in this example, to facilitate description, it is assumed that sets of two signals are averaged. In other words, the S/N ratio is improved by averaging two sets of photoacoustic signals obtained in response to two light emission operations.

More specifically, in Td, digital signals having identical numbers (D1 and D1′, D2 and D2′, D3 and D3′, . . . ) are averaged, and an image is reconstructed on the basis of the resulting averaged photoacoustic signals. As a result, a reconstructed image having reduced noise can be acquired.

By averaging the photoacoustic signals in this manner, thermal noise and Schottkey noise generated in a circuit such as a transducer or an amplifier can be reduced. These types of noise are generated at substantially random timings, and therefore the noise can be reduced by executing averaging.

However, noise intermixed in the photoacoustic signal includes external noise intermixed in an analog circuit between the probe and the A/D converter as well as noise generated in the interior of the device. External noise is switching noise from a switching power supply, noise from a motor controller and a motor, noise generated on the basis of the clock of a digital circuit or the like, and so on, for example. These types of noise, in contrast to thermal noise and Schottkey noise, are typically generated periodically. External noise is generated inside or outside the photoacoustic device, and may become intermixed in the aforementioned analog circuit. It is difficult to eliminate noise intermixing from the outside from the photoacoustic device.

Tn in FIG. 6A denotes an example of external noise input into the A/D converter. S and S′ are waveforms schematically representing the external noise. This example shows a case in which the sampling period is set at 0.1 milliseconds, and the external noises S and S′ are likewise generated every 0.1 milliseconds (at 10 kHz).

Here, the A/D conversion clock is set at 40 MHz (a period of 25 nanoseconds), for example. The A/D converter converts an input analog signal S1 into a digital signal D6, and likewise converts a signal S2, a signal S3, and a signal S4 into a signal D7, a signal D8, and a signal D9, respectively. In the next sampling period, meanwhile, the A/D converter converts an input analog signal S1′ into a digital signal D6′, and likewise converts a signal S2′, a signal S3′, and a signal S4′ into a signal D7′, a signal D8′, and a signal D9′, respectively.

The digital signal D6 and the digital signal D6′, the signal D7 and the signal D7′, the signal D8 and the signal D8′, and the signal D9 and the signal D9′ are then respectively averaged.

Needless to mention, however, the periodic external noises S and S′ indicated by Tn are not reduced by averaging.

In this example, a case in which the sampling period is identical to the external noise generation period is shown, but when a repetition frequency of the external noise is an integral multiple of the sampling period, noise is generated at an identical timing in each sampling period on the basis of a light emission control signal. As described above, therefore, noise cannot be suppressed by averaging.

Further, when the sampling frequency is comparatively low, the repetition frequency of the external noise is more likely to be an integral multiple of the sampling frequency. For example, noise generated by the switching power supply (from 10 kHz to several hundred kHz) may match this frequency. Hence, the number of types of external noise that cannot be suppressed by averaging is comparatively large.

The photoacoustic device according to the first embodiment is capable of suppressing this periodically generated external noise. Referring to FIG. 6B, a method for reducing periodic external noise will be described.

The example shown in FIG. 6B differs from FIG. 6A in that the interval between adjacent sampling timings is not constant. In other words, the photoacoustic device according to the first embodiment emits light and acquires digital signals at non-periodic sampling timings.

As indicated by T1 in FIG. 6B, a sampling interval tw1− is shorter than the sampling interval tw1 by four cycles of the A/D conversion clock. Further, a sampling interval tw1+ is longer than the sampling interval tw1 by four cycles of the A/D conversion clock.

The photoacoustic wave coming from the subject is generated using the light emission control signal as a trigger, and therefore, even when the sampling timing is shifted, the acquired photoacoustic signal (digital signal) is identical to that of FIG. 6A.

Meanwhile, the external noise is subjected to analog/digital conversion in the following manner. The sampling interval tw1− is four cycles short, and therefore a digital signal generated when the external noise S′ is subjected to A/D conversion at a second sampling timing is retarded by four cycles.

More specifically, the input analog signal S1′ is converted into a digital signal D10′, and similarly, the signal S2′, the signal S3′, and the signal S4′ are converted into a signal D11′, a signal D12′, and a signal D13′, respectively. The digital signal D6 and the digital signal D6′, the signal D7 and the signal D7′, the signal D8 and the signal D8′, . . . , and the signal D13 and the signal D13′ are then respectively averaged.

As a result, the amplitude of the external noise is halved. Moreover, by increasing the number of averaged signals, the external noise can be further reduced.

As described above, the photoacoustic device according to this embodiment implements averaging in a state where the sampling interval (sampling period) for acquiring the photoacoustic signals is variable. As a result, external noise can be reduced while ensuring that the photoacoustic signals do not deteriorate.

To reduce external noise, the sampling interval is preferably varied randomly. Further, a minimum duration of the sampling interval is preferably determined on the basis of a value obtained by dividing a maximum value of a distance value between the transducer and a light absorber by an acoustic velocity in the interior of the subject. For example, when a photoacoustic wave generated from a light absorber located 15 cm away from the transducer is to be received, assuming that the acoustic velocity of a human body is 1500 m/sec, the minimum duration of the sampling interval is set at 0.1 milliseconds. In this case, the sampling interval is preferably varied randomly within a width of ±0.02 milliseconds about a sampling interval of 0.12 milliseconds.

Alternatively, when the number of averaged signals is 41, for example, control may be implemented to increase the sampling interval by 0.001 milliseconds in each sampling operation from 0.1 milliseconds up to 0.14 milliseconds. In this case, the sampling interval is increased monotonically with each sampling operation so as to vary in a saw tooth-shaped waveform. Conversely, the sampling interval may be reduced monotonically. Hence, the sampling interval may be varied using a method other than random variation. A similar external noise reduction effect is obtained even when the sampling interval is not varied randomly.

Further, when the sampling interval is modified, the sampling interval is preferably determined on the basis of the A/D conversion clock. By generating the light emission control signal on the basis of the A/D conversion clock, the light emission and A/D conversion timings can be fixed. In other words, jitter can be removed from a single period of the A/D conversion clock, and as a result, a more favorable reconstructed image can be acquired.

In this case, a circuit for modifying the sampling interval is preferably realized using a programmable counter having the A/D conversion clock as an input. More specifically, the circuit can be realized by setting a number of A/D conversion clocks corresponding to the sampling interval in a register of the programmable counter for each sampling interval. The programmable counter compares the value in the register with a count value, and when the values match, outputs a signal for clearing the count value to zero. Hence, the clear signal is preferably used as the light emission control signal.

For example, when the A/D conversion clock is set at 40 MHz, a set value of the register for setting the sampling period at 0.1 milliseconds is 4000. By setting the value in the register of the programmable counter having the A/D conversion clock as an input in a timely fashion (using a computer 150, for example) in this manner, a desired sampling interval can be realized.
<Device configuration>

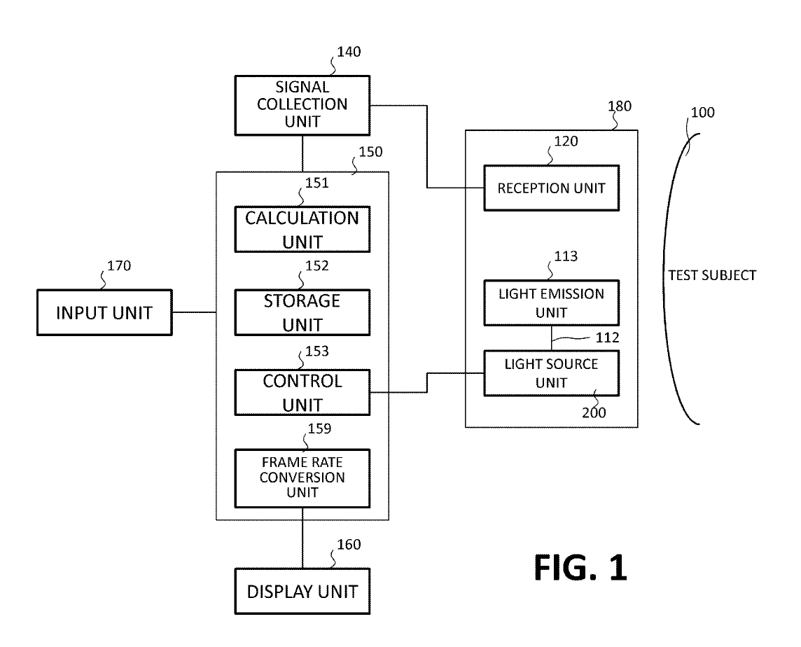

A configuration of the photoacoustic device according to the first embodiment will be described below with reference to FIG. 1. The photoacoustic device according to the first embodiment is configured to include a probe 180, a signal collection unit 140, the computer 150, a display unit 160, and an input unit 170. The probe 180 includes a light source unit 200, an optical system 112, a light emission unit 113, and a reception unit 120. The computer 150 includes a calculation unit 151, a storage unit 152, a control unit 153, and a frame rate conversion unit 159.

Here, a method for performing measurements on the subject will be described briefly.

First, the light source unit 200 supplies pulsed light to the light emission unit 113 via the optical system 112, which is constituted by optical fiber (bundled fiber) or the like. Further, the light emission unit 113 emits the supplied light to the subject 100.

The reception unit 120 receives a photoacoustic wave generated by the subject 100, and outputs an analog electric signal. The signal collection unit 140 converts the analog signal output from the reception unit 120 into a digital signal, and outputs the digital signal to the computer 150.

As noted above, the pulsed light is emitted at a non-periodic sampling timing, or in other words a non-constant sampling interval. Therefore, the electric signal corresponding to the acoustic wave generated in response to the pulsed light is likewise output in time series at each sampling interval.

The computer 150 executes processing to average the digital signals output from the signal collection unit 140 at the respective sampling timings at each period (referred to hereafter as an imaging cycle; a second period according to the present invention) corresponding to an imaging frame rate, and stores the resulting digital signals in the memory. The computer 150 then executes image reconstruction processing on the stored digital signals so as to generate photoacoustic image data.

Further, the computer 150 outputs the acquired photoacoustic image data to the frame rate conversion unit 159 at each imaging cycle. The frame rate conversion unit 159 converts the frame rate of the photoacoustic image data input therein at each imaging cycle into a refresh rate (referred to hereafter as a display cycle; a third period according to the present invention) corresponding to the display unit 160. A detailed method will be described below.

The display unit 160 then displays the photoacoustic image data while refreshing the data at each display cycle.

A user of the device (a doctor, a technician, or the like) can implement a diagnosis by checking the photoacoustic image displayed on the display unit 160. The displayed image may be stored in the memory of the computer 150, a data management system connected to the photoacoustic device by a network, or the like on the basis of a store instruction from the user or the computer 150. The user of the device can perform input on the device via the input unit 170.

Next, the respective constituent elements will be described in detail.
Probe 180

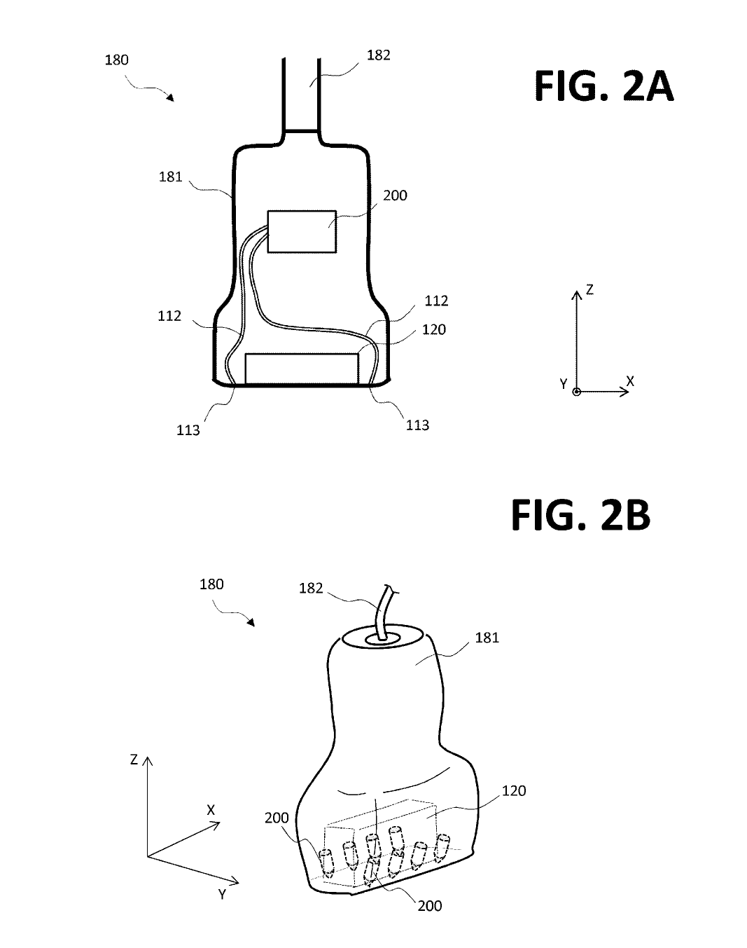

FIG. 2A is a schematic view of the probe 180 according to this embodiment. The probe 180, which also forms a part of an acoustic wave detection unit, includes the light source unit 200, the optical system 112, the light emission unit 113, the reception unit 120, and a housing 181.

The housing 181 houses the light source unit 200, the optical system 112, the light emission unit 113, and the reception unit 120. The user can use the probe 180 as a handheld probe by gripping the housing 181.

The light emission unit 113 serves as means for emitting pulsed light, propagated by the optical system 112, to the subject. Note that XYZ axes in the figure denote coordinate axes in a case where the probe is stationary, and do not limit the orientation of the probe while in use.

The probe 180 shown in FIG. 2A is connected to the signal collection unit 140 via a cable 182. The cable 182 includes a wire for supplying power to the light source unit 200, a wire for transmitting the light emission control signal to the light source unit 200, a wire for outputting an analog signal output from the reception unit 120 to the signal collection unit 140, and so on (none of which are shown in the figure). Note that the cable 182 may be provided with a connector so that the probe 180 can be attached to and detached from another configuration of the photoacoustic device.

Further, as shown in FIG. 2B, a semiconductor laser, a light-emitting diode, or the like may be used as the light source unit 200, and the subject may be irradiated with pulsed light directly, without using the optical system 112. In this case, a light-emitting end part (a tip end of the housing) constituted by a semiconductor laser, an LED, or the like serves as the light emission unit 113.
<Light source unit 200>

The light source unit 200 serves as means for generating the light to be emitted to the subject 100.

The light source is preferably a laser light source so that a large output is obtained, but a light-emitting diode, a flash lamp, or the like may be used instead of a laser. When a laser is used as the light source, various types of lasers, such as a solid-state laser, a gas laser, a dye laser, or a semiconductor laser, may be used. The timing, waveform, intensity, and so on of emission is controlled by a light source control unit, not shown in the figures. This light source control unit may be integrated with the light source.

Further, when a substance concentration such as the oxygen saturation is to be acquired, a light source capable of outputting a plurality of wavelengths is preferably used. Furthermore, when the light source unit 200 is packaged in the housing 181, a semiconductor light-emitting element such as a semiconductor laser or a light-emitting diode is preferably used, as shown in FIG. 2B. Moreover, when a plurality of wavelengths are output, the wavelength may be switched by employing a plurality of types of semiconductor lasers or light-emitting diodes that generate light of different wavelengths.

To generate a photoacoustic wave effectively, light must be emitted for a sufficiently short time in accordance with a thermal characteristic of the subject. When the subject is a living organism, the pulse width of the pulsed light generated by the light source is preferably between approximately 10 nanoseconds and 1 microsecond. Further, the wavelength of the pulsed light is preferably set such that the light propagates to the interior of the subject. More specifically, when the subject is a living organism, the wavelength is preferably set to be at least 400 nm and not more than 1600 nm. Needless to mention, the wavelength may be determined in accordance with a light absorption characteristic of the light absorber to be subjected to imaging.

Note that when a blood vessel is to be imaged at a high resolution, a wavelength (at least 400 nm and not more than 800 nm) at which the blood vessel absorbs a large amount of light may be used. Further, when a deep part of a living organism is to be imaged, light having a wavelength (at least 700 nm and not more than 1100 nm) at which a small amount of light is absorbed by background tissue (water, fat, and so on) of the living organism may be used.

In this embodiment, a semiconductor light-emitting element is used as the light source, and therefore the subject cannot be irradiated with a large quantity of light. In other words, a photoacoustic signal acquired from a single emission is unlikely to reach a desired S/N ratio. Therefore, the S/N ratio is improved by having the light source emit light at non-periodic sampling timings, and averaging the generated photoacoustic signals.

797 nm may be cited as an example of a favorable wavelength of the light source unit 200 used in this embodiment. This wavelength is large enough to reach a deep part of the subject, and exhibits substantially equal oxyhemoglobin and deoxyhemoglobin absorption coefficients so as to be suitable for detecting a blood vessel structure. Moreover, by employing 756 nm as a second wavelength, the oxygen saturation can be determined using a difference between the oxyhemoglobin and deoxyhemoglobin absorption coefficients.
<Light emission unit 113>

The light emission unit 113 is a site (an emission end) from which to emit the light with which the subject is irradiated. When bundled fiber is used as the optical system 112, a terminal end portion thereof serves as the light emission unit 113. Moreover, a diffusion plate or the like for diffusing the light may be disposed on the light emission unit 113. In so doing, the subject can be irradiated with the pulsed light after widening a beam diameter thereof.

Furthermore, when a plurality of semiconductor light-emitting elements are used as the light source unit 200, as shown in FIG. 2B, by arranging the light-emitting end parts (the housing tip ends) of the respective elements so as to form the light emission unit 113, the subject can be irradiated over a wider range.
<Reception unit 120>

The reception unit 120 is constituted by a transducer (an acoustic wave detection element) that outputs an electric signal after receiving the photoacoustic wave generated in response to the pulsed light, and a support that supports the transducer.

A piezoelectric material, an electrostatic capacitance type transducer (a CMUT), a transducer employing a Fabry-Perot interferometer, and so on may be cited as examples of members constituting the transducer. Further, a piezoelectric ceramic material such as PZT (lead zirconate titanate) and a piezoelectric polymer film material such as PVDF (polyvinylidene fluoride) may be cited as examples of the piezoelectric material.

The electric signal acquired by the transducer is a time-resolved signal. In other words, the amplitude of the acquired electric signal takes a value based on an acoustic pressure received by the transducer at each time interval (for example, a value that is proportionate to the acoustic pressure).

Note that a transducer capable of detecting a frequency component (typically from 100 kHz to 10 MHz) of the photoacoustic wave is preferably used as the transducer. Furthermore, a plurality of transducers may be arranged on the support to form a planar surface or a curved surface known as a 1D array, a 1.5D array, a 1.75D array, or a 2D array.

Further, the reception unit 120 may include an amplifier for amplifying the time-series analog signals output by the transducer. The reception unit 120 may also include an A/D converter for converting the time-series analog signals output by the transducer into time-series digital signals. In other words, the reception unit 120 may double as the signal collection unit 140.

In this embodiment, a handheld probe has been described as an example, but to improve the image precision, a transducer that surrounds the subject 100 from the entire periphery thereof is preferably used so that acoustic waves can be detected from various angles. Further, when the subject 100 is too large for the transducer to surround the entire periphery thereof, the transducer may be disposed on a hemispherical support. When the probe includes a reception unit having this shape, the probe can be moved mechanically relative to the subject 100. A mechanism such as an XY stages can be used to move the probe. Note that the position of the transducer, the number of transducers, and the shape of the support are not limited to those described above, and may be optimized in accordance with the subject 100.

A medium (an acoustic matching material) through which the photoacoustic wave propagates is preferably disposed between the reception unit 120 and the subject 100. In so doing, acoustic impedance on an interface between the subject 100 and the transducer can be matched. Water, oil, ultrasound gel, and so on, for example, may be used as the acoustic matching material.

Furthermore, the photoacoustic device according to this embodiment may include a holding member for holding the subject 100 so as to stabilize the shape thereof. A member exhibiting superior light transmission and acoustic wave transmission properties is preferably used as the holding member. For example, polymethylpentene, polyethylene terephthalate, acrylic, or the like can be used.

When the device according to this embodiment has a function for generating an ultrasound image by transmitting and receiving ultrasonic waves in addition to a photoacoustic image, the transducer may be caused to function as transmitting means for transmitting acoustic waves. The transducer serving as the receiving means and the transducer serving as the transmitting means may be constituted by a single transducer or separate transducers.
<Signal collection unit 140>

The signal collection unit 140, which also forms a part of the acoustic wave detection unit, includes an amplifier for amplifying the analog electric signal output from the reception unit 120, and an A/D converter for converting the analog signal output from the amplifier into a digital signal. The signal collection unit 140 may be constituted by a field programmable gate array (FPGA) chip or the like.

Analog signals output by the plurality of transducers arrayed on the reception unit 120 are amplified by a plurality of amplifiers corresponding respectively thereto, and converted into digital signals by a plurality of A/D converters corresponding respectively thereto. A rate of the A/D converter is preferably at least twice the bandwidth of the input signal. When the frequency component of the photoacoustic wave is between 100 kHz and 10 MHz, as noted above, the A/D conversion rate is at least 20 MHz, and preferably at least 40 MHz.

As described above, the signal collection unit 140 uses the light emission control signal to synchronize the light emission timing with the signal collection processing timing. In other words, the signal collection unit 140 converts the analog signals into digital signals by starting A/D conversion at the aforementioned A/D conversion rate using the light emission timing, which is a non-periodic sampling timing, as a reference. As a result, a sequence of digital signals can be acquired by each transducer over a single interval (the period of the A/D conversion clock) corresponding to the A/D conversion rate. In other words, photoacoustic signals based on the light emission timing can be acquired accurately even when the sampling timing is non-periodic. The signal collection unit 140 is also known as a data acquisition system (DAS).

As noted above, the signal collection unit 140 may be disposed in the housing 181 of the probe 180. With this configuration, information can be propagated between the probe 180 and the computer 150 by digital signals, leading to an improvement in noise resistance. Further, in comparison with a case where analog signals are transmitted, fewer wires are required, and therefore the operability of the probe 180 is improved. Moreover, the averaging to be described below may also be executed by the signal collection unit 140. In this case, the averaging is preferably executed using hardware such as an FPGA.
<Computer 150>

The computer 150 serves as calculating means including the calculation unit 151 (an image generation unit according to the present invention), the storage unit 152, the control unit 153, and the frame rate conversion unit 159. Units for realizing the calculation functions of the calculation unit 151 may be constituted by a processor such as a CPU or a graphics processing unit (GPU), and a calculation circuit such as a field programmable gate array (FPGA) chip. These units may be formed from a single processor and a single calculation circuit, or pluralities of processors and calculation circuits.

The computer 150 executes the following processing on each of the plurality of transducers.

First, the computer 150 adds together and averages contemporaneous data acquired at the same light emission timing in relation to the digital signals output from the signal collection unit 140 at each sampling timing. The averaged digital signals are then stored in the storage unit 152 in each imaging cycle as averaged photoacoustic signals.

The calculation unit 151 then executes image reconstruction on the basis of the (averaged) photoacoustic signals stored in the storage unit 152 in order to generate a photoacoustic image (a structural image or a functional image), and executes other calculation processing. Note that the calculation unit 151 may receive various parameter inputs relating to the acoustic velocity through the interior of the subject, the structure of the holding portion, and so on from the input unit 170, and use these parameters in the calculations.

Any desired method, such as a time-domain back projection method, a Fourier-domain back projection method, or a model-based method (a repetitive operation method), may be used by the calculation unit 151 as a reconstruction algorithm for converting the photoacoustic signals into a photoacoustic image (three-dimensional volume data, for example). Universal back projection (UBP), filtered back projection (FBP), phasing addition (delay and sum), and so on may be cited as time-domain back projection methods.

When the light source unit 200 generates light having two different wavelengths, during the image reconstruction processing, the calculation unit 151 generates a first initial acoustic pressure distribution and a second initial acoustic pressure distribution from photoacoustic signals derived from light having a first wavelength and from photoacoustic signals derived from light having a second wavelength, respectively. Further, the calculation unit 151 acquires a first absorption coefficient distribution by correcting the first initial acoustic pressure distribution using a light quantity distribution of the light having the first wavelength, and acquires a second absorption coefficient distribution by correcting the second initial acoustic pressure distribution using a light quantity distribution of the light having the second wavelength. Furthermore, the calculation unit 151 acquires the oxygen saturation distribution from the first and second absorption coefficient distributions. Note that as long as the oxygen saturation distribution can eventually be acquired, the content and sequence of the calculations are not limited to those described above.

The storage unit 152 is constituted by a volatile memory such as a random access memory (RAM), or a non-temporary storage medium such as a read only memory (ROM), a magnetic disc, or a flash memory. Note that a non-temporary storage medium is used as a storage medium for storing a program. The storage unit 152 may be constituted by a plurality of storage media.

Various data, such as the photoacoustic signals averaged in the respective imaging cycles, the photoacoustic image data generated by the calculation unit 151, and reconstructed image data based on the photoacoustic image data, can be stored in the storage unit 152. Further, when it is possible to set a plurality of sampling interval variation patterns, the patterns (random variation, monotonic increase, monotonic reduction, and so on) and data (the value in the register of the programmable counter having the A/D conversion clock as an input, and so on, for example) relating respectively thereto can also be stored in the storage unit 152.

The control unit 153 serves as means for controlling operations of the respective constituent elements of the photoacoustic device, and is constituted by a calculation element such as a CPU. The control unit 153 may control the respective constituent elements of the photoacoustic device on the basis of instruction signals (a measurement start signal and so on, for example) input via the input unit 170.

Further, the control unit 153 controls the operations of the respective constituent elements of the photoacoustic device by reading program code stored in the storage unit 152. As described above, regardless of the manner in which the sampling interval is varied, the sampling intervals can be realized using a programmable counter having the A/D conversion clock as an input. The control unit 153 can set the interval between adjacent sampling timings at a desired interval by setting the value in the register of the programmable counter.

Furthermore, at this time, by setting a sum of the duration of a plurality of sampling intervals not to exceed the imaging cycle, averaging can be executed within the imaging cycle. Note that when the sum of the duration exceeds the imaging cycle, the averaged data partially overlap, but averaging remains possible, and therefore the effects of the present invention are still obtained.

The control unit 153 is also capable of adjusting the generated image and so on.

The frame rate conversion unit 159 serves as means for converting photoacoustic images generated at a predetermined frame rate (the imaging frame rate) corresponding to the imaging cycle into a predetermined frame rate (referred to hereafter as a display frame rate) corresponding to the display cycle, and outputting the converted images to the display unit 160.

Note that in the example shown in FIG. 1, the frame rate conversion unit 159 is configured independently, but the frame rate conversion unit 159 does not have to be configured independently. Instead, for example, photoacoustic images may be stored in the storage unit 152 in accordance with the imaging frame rate, and the stored photoacoustic images may be read in accordance with the display frame rate.

In the present invention, even when the frame rate is converted using another method, the corresponding part is known as the frame rate conversion unit.

The display frame rate is preferably set at a frame rate (50 Hz, 60 Hz, 72 Hz, 120 Hz, or the like, for example) corresponding to a general-purpose display. By setting the imaging cycle and the display cycle independently of each other in this manner, a suitable frame rate for measurement and a suitable frame rate for image display can be set individually. In other words, a suitable frame rate for measurement can be set freely, without taking into consideration a suitable frame rate for image display. Moreover, the imaging cycle can be freely modified alone in response to an instruction from the user, for example.

The display unit 160 serves as means for displaying photoacoustic images. The display unit 160 rewrites an actual screen in synchronization with the display frame rate. Note that the display frame rate and the rate (the refresh rate) at which the actual screen is rewritten may be identical.

Some recent liquid crystal displays have a function for handling input at a plurality of frame rates (frame frequencies). Some of these liquid crystal displays have a function for converting the input frame rate into the rate (the refresh rate) at which the actual screen is rewritten. When the display unit 160 has these functions, it may be said that the display unit 160 has an inbuilt frame rate converter for converting the display frame rate into the actual refresh rate.

Further, when this type of display unit 160, i.e. a display unit having an inbuilt frame rate converter, is used, there is no need to provide the frame rate conversion unit 159 shown in FIG. 1 in the computer 150. By providing the functions of the frame rate conversion unit in the display unit 160, the configuration of the computer 150 can be simplified.

Furthermore, a configuration for converting the display frame rate into the refresh rate is not an essential configuration. When the two frame rates are identical, for example, the frame rate conversion unit 159 can be omitted. Needless to mention, the object of the present invention, i.e. to reduce external noise, can be realized in this case also.

The computer 150 may be a specially designed work station or a general-purpose PC or work station. The computer 150 may be operated in accordance with instructions from the program stored in the storage unit 152. Further, the respective configurations of the computer 150 may be formed from different pieces of hardware. Furthermore, at least some of the configurations of the computer 150 may be formed from a single piece of hardware.

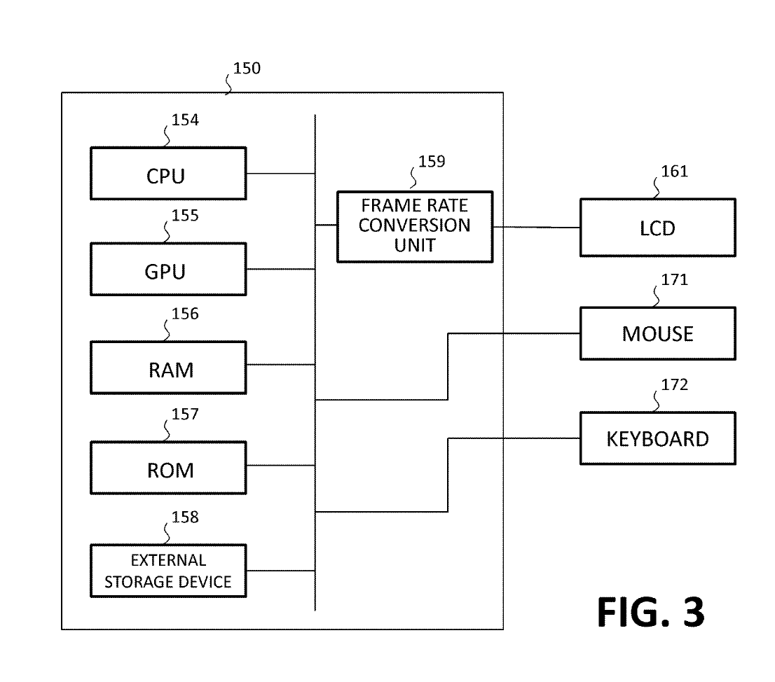

FIG. 3 shows a specific example configuration of the computer 150 according to this embodiment. The computer 150 according to this embodiment includes a CPU 154, a GPU 155, a RAM 156, a ROM 157, an external storage device 158, and the frame rate conversion unit 159. Further, a liquid crystal display 161 serving as the display unit 160 and a mouse 171 and a keyboard 172 serving as the input unit 170 are connected to the computer 150.

The computer 150 and the reception unit 120 may be housed in a common housing. Further, a part of the signal processing may be executed by the computer housed in the housing, and the remainder of the signal processing may be executed by a computer provided on the exterior of the housing. In this case, the computers provided respectively inside and outside the housing together constitute the computer according to this embodiment. In other words, the hardware forming the computer may be dispersed. Furthermore, an information processing device disposed in a remote location and provided by a cloud computing service or the like may be used as the computer 150.

Note that the computer 150 may execute image processing on the acquired photoacoustic images and processing for synthesizing GUI graphics and so on therewith as required. Moreover, this processing may be executed before or after frame rate conversion.
<Display unit 160>

The display unit 160 is a display device such as a liquid crystal display or an organic EL. The display unit 160 displays images generated by the computer 150, and displays numerical values and the like in specific positions. As described above, images are input into the display unit 160 at a frame rate (50 Hz, 60 Hz, 72 Hz, 120 Hz, or the like, for example) corresponding to the display cycle. The display unit 160 may display the images at the input frame rate, or may further convert the frame rate. The display unit 160 may also display a GUI used to manipulate the images and operate the device on the screen.
<Input unit 170>

The input unit 170 serves as means for acquiring input such as instructions and numerical values from the user. The user can start and stop measurement, specify the sampling interval variation pattern, issue an instruction to store a generated image, and so on via the input unit 170.

The input unit 170 may be, for example, an operating console constituted by a mouse, a keyboard, a dedicated button, and so on that can be operated by the user. Note that by employing a touch panel as the display unit 160, the display unit 160 can double as the input unit 170.

The constituent elements of the photoacoustic device, as described above, may be constituted respectively by separate devices, or may all be integrated. Alternatively, at least some of the configurations of the photoacoustic device may be integrated, and the remaining configurations may be constituted by separate devices.
<Subject 100>

The subject 100, although not a part of the photoacoustic device according to the present invention, will now be described. The photoacoustic device according to this embodiment can be used to diagnose a malignant tumor, a vascular disease, and so on in a human or animal, to observe a course of chemotherapy, and so on. Hence, a living organism, and more specifically a diagnosis target site such as a breast, an organ, the vascular network, the head, the neck, the abdomen, or an extremity such as a finger or a toe of a human or animal is envisaged as the subject 100. For example, when the measurement subject is a human body, for instance, a blood vessel containing large amounts of oxyhemoglobin and deoxyhemoglobin, and a new blood vessel formed in the vicinity of a tumor may be set as a light absorption subject. Further, plaque or the like on the carotid wall may be set as the light absorption subject. Moreover, a dye such as methylene blue (MB) or indocyanine green (ICG), metal particles, or a substance that is obtained by aggregating or chemically modifying these substances and introduced from the outside may be used as the light absorber. Furthermore, a puncture needle or a light absorber attached to a puncture needle may be used as an observation subject. The subject may also be an inanimate object such as a phantom or a product under test.
<Details of processing>

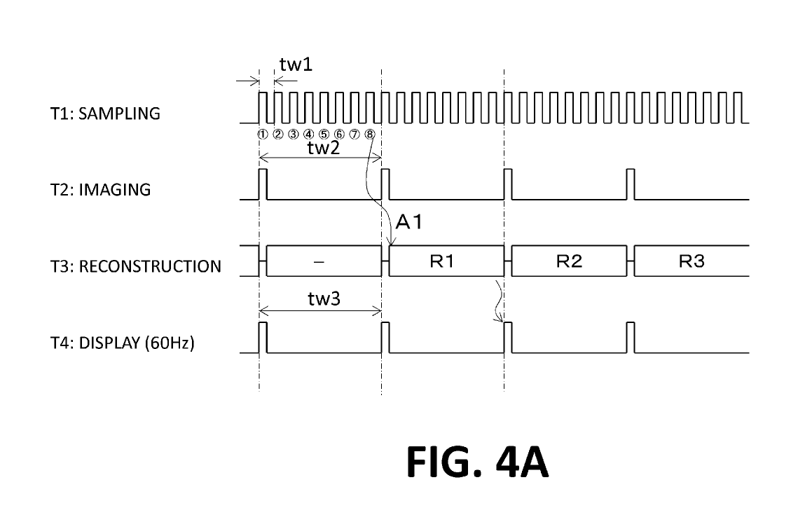

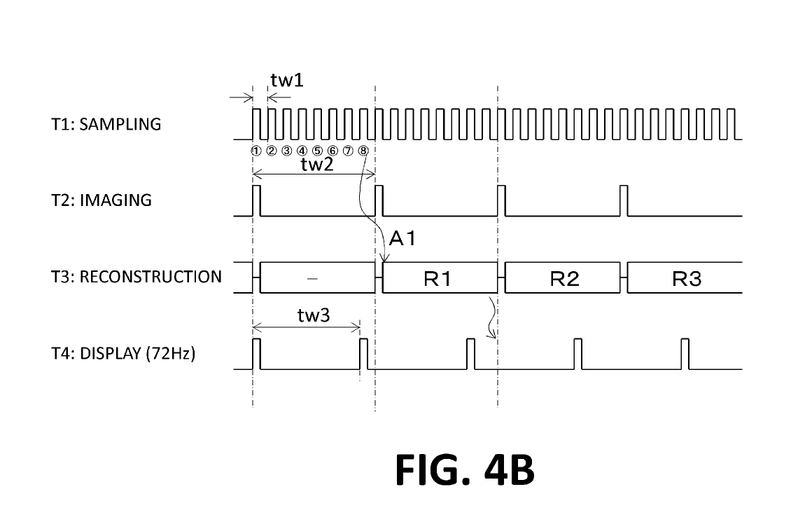

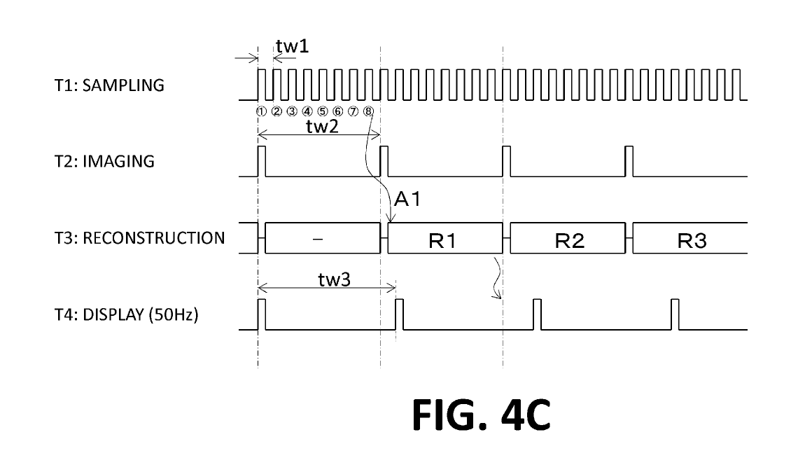

Next, the processing will be described in detail with reference to FIGS. 4A to 4C, which are timing diagrams illustrating operations of the photoacoustic device according to the first embodiment. Note that in each of the figures, the horizontal axis is a temporal axis.

First, referring to FIG. 4A, a method for acquiring photoacoustic signals and a method for generating a photoacoustic image on the basis of the acquired photoacoustic signals will be described. Note that to facilitate description, the imaging frame rate and the display frame rate are set to be identical in the example shown in the figure.

As indicated by T1 in FIG. 4A, in the photoacoustic device according to this embodiment, the light source unit 200 emits light at sampling intervals (tw1), which are intervals between non-periodic sampling timings, whereby photoacoustic signals generated in response to emission of the light are acquired at intervals of the sampling timing. Although not indicated explicitly in the figure, the sampling intervals tw1 are different from each other.

Note that the length of the sampling interval tw1 may be set in consideration of the maximum permissible exposure (MPE) to the skin. For example, when the measurement wavelength is 750 nm, the pulse width of the pulsed light is 1 microsecond, and the sampling interval tw1 is 0.1 milliseconds, the MPE value relative to skin is approximately 14 J/m². Meanwhile, when the peak power of the pulsed light emitted from the light emission unit 113 is 2 kW and an emission area from the light emission unit 113 is 150 mm², the subject 100 is irradiated with approximately 13.3 J/m² of optical energy. In this case, the optical energy emitted from the light emission unit 113 does not exceed the MPE value.

Hence, even when the sampling interval varies, as long as the sampling interval tw1 satisfies a condition of being no shorter than 0.1 milliseconds, the optical energy can be prevented from exceeding the MPE value. Thus, the optical energy with which the subject is irradiated can be calculated using the value of the sampling interval tw1, the peak power of the pulsed light, and the emission area.

It is assumed here that eight photoacoustic signals are acquired in time series at each sampling timing and averaged. Here, an averaged photoacoustic signal A1 is acquired in each imaging cycle tw2 (T2). Note that a simple average, a moving average, a weighted average, or the like may be used as the average. For example, when an average value of the sampling interval tw1 is 0.1 milliseconds and the imaging frame rate is 60 Hz, tw2 is 16.7 milliseconds, and therefore 167 signals can be averaged within the period of the imaging frame rate.

Next, the reconstruction processing described above is executed on the basis of the averaged photoacoustic signal A1 in order to determine reconstructed image data R1 (T3). The image data are generated successively in each imaging cycle.

As noted above, in this example, the imaging frame rate and the display frame rate are identical. Hence, the frame rate conversion unit 159 outputs the image data R1 generated in T3 within a period (the display cycle) tw3 corresponding to the display frame rate. The display unit 160 then displays the image data input in the display cycle tw3.

A method for determining the sampling interval tw1 and the imaging cycle tw2 will now be described.

As noted above, the minimum value of the sampling interval tw1 is determined on the basis of a limitation caused by the MPE value. Further, the number of averaged signals is determined by the S/N ratio of the photoacoustic signal acquired from a single emission of pulsed light and an S/N ratio for acquiring a required image quality.

For example, when the S/N ratio of the photoacoustic signal acquired from a single emission of pulsed light is one tenth of the required S/N ratio, the S/N ratio must be multiplied by ten. Accordingly, 100 signals must be averaged. For example, when the average value of the sampling interval tw1 is 0.1 milliseconds, the imaging cycle must be set to be at least 10 milliseconds. In other words, the imaging frame rate must not exceed 100 Hz.

Note that the average value of the sampling interval tw1 is also limited due to heat generation by the semiconductor light-emitting element. In other words, the average value of the sampling interval tw1 must be lengthened so that the temperature of the semiconductor light-emitting element does not exceed an allowable temperature.

On the other hand, when the number of averaged signals is increased, the photoacoustic signals are averaged over a long period of time, and as a result, blur caused by movement of the subject occurs. Minimizing the number of averaged signals is effective in reducing motion blur. More specifically, the photoacoustic device is preferably designed so that motion blur is suppressed to or below ½ the required resolution. For example, when the required resolution is 0.2 milliseconds, movement of the subject is 5 millimeters per second, and a maximum value of the sampling interval tw1 is 0.2 milliseconds, the number of averaged signals should not to exceed 100, or in other words the imaging cycle tw2 should not exceed 20 milliseconds.

The average value of the sampling interval tw1 and the imaging cycle tw2 should be determined in consideration of the plurality of conditions described above. Moreover, when it is impossible to satisfy all of these conditions, the parameters may be determined after setting degrees of priority.

FIGS. 4B and 4C show an example of a case in which the imaging frame rate and the display frame rate are different. The example in FIGS. 4B and 4C differs from the example in FIG. 4A only in that a display frame rate T4 is different. In other words, under identical measurement conditions to those of the example in FIG. 4A, identical reconstructed image data can be acquired.

FIG. 4B shows an example in which the display frame rate (T4) has been modified from 60 Hz to 72 Hz. In other words, the display cycle tw3 is approximately 13.8 milliseconds. FIG. 4C, meanwhile, shows an example in which T4 has been modified from 60 Hz to 50 Hz. In other words, the display cycle tw3 is 20 milliseconds.

As described above, the reconstructed image data are converted by the frame rate conversion unit 159 from the imaging frame rate (60 Hz, for example) to the display frame rate (72 Hz or 50 Hz, for example). The frame rate can be converted by frame pruning or overwriting. When the probe moves quickly such that a sense of interference becomes noticeable, the frame rate is preferably converted by, for example, implementing inter-frame interpolation using a motion vector or the like to generate an interpolation frame.

In the first embodiment, as described above, in the photoacoustic device that averages photoacoustic signals, the timings at which light is emitted and photoacoustic signals are acquired are set to be non-periodic. In so doing, periodic external noise other than random noise can be reduced. As a result, the image quality of the acquired reconstructed image can be improved.

### Second Embodiment

In the first embodiment, a time obtained by multiplying the number of averaged signals by the average value of the duration of the sampling interval tw1 must be identical to the imaging cycle, and therefore setting of the sampling interval is limited. In a second embodiment, this limitation is avoided by providing a sampling rest period.

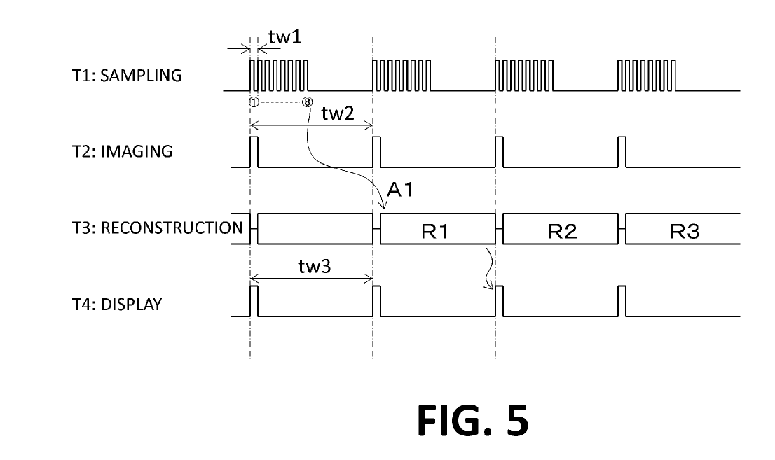

FIG. 5 is a timing diagram pertaining to the second embodiment. The example in FIG. 5 differs from the example in FIG. 4A only in the sampling interval T1. In other words, operation timings from T2 to T4 are identical.

A photoacoustic device according to the second embodiment is designed such that a time obtained by multiplying the number of averaged signals by the maximum value of the sampling interval tw1 is shorter than the imaging cycle, with the remaining time being set as a rest period.

By designing the photoacoustic device in this manner, all of the sampling clocks can be accommodated within the imaging cycle. In other words, light emission and photoacoustic signal acquisition can be completed within the imaging cycle. By designing the photoacoustic device in this manner, conditions on the manner in which the sampling interval is varied can be alleviated.

In the second embodiment, as described above, by providing the rest period in addition to the first embodiment, an improvement in the setting freedom of the sampling interval can be achieved.

### Third Embodiment

In a third embodiment, sampling timings of a series of sampling operations (i.e. the variation pattern of the sampling interval) are defined in advance so as to be selectable by the user.

Examples of sampling interval variation patterns include random variation, monotonic increase, and monotonic reduction. In the third embodiment, the user or a technician who disposes the photoacoustic device can select and set the pattern to be employed while viewing a reconstructed image displayed on the display unit 160. As a result, it is possible to select a pattern with which noise generated in the environment in which the photoacoustic is disposed can be favorably reduced.

Note that in order to select a pattern, preferably, measurement is implemented without providing a subject (in other words, without generating photoacoustic waves), and the reconstructed image acquired as a result is displayed on the display unit 160. For example, the reconstructed image is preferably acquired in a condition where no subject exists and light emission from the light source unit 200 is prohibited. External noise may vary according to the location in which the photoacoustic device is disposed and the condition of other devices adjacent thereto, and therefore, in so doing, it is possible to select a pattern with which external noise can be effectively suppressed.

### Other Embodiments

Note that the embodiments described above are examples used to illustrate the present invention, and the present invention may be implemented by appropriately modifying or combining the embodiments within a scope that does not depart from the spirit thereof.

For example, the present invention may be implemented in the form of a subject information acquisition device that executes at least a part of the processing described above. Further, the present invention may be implemented in the form of a subject information acquisition method including at least a part of the processing described above. The processing and means described above may be combined freely and implemented thus, providing that no technical contradictions arise as a result.

Furthermore, in the embodiments described above, the terms “first period” (the sampling interval), “second period” (the imaging cycle), and “third period” (the display cycle) were used, but these periods do not necessarily have to be perfectly constant. In other words, the term “period” as used in this specification includes repetition of non-constant time intervals. Moreover, as described above, a rest period may be provided in the first period (the sampling interval). In the present invention, a repeated time period not including a rest period is referred to as a period.

Further, as described above, the light source unit 200 may emit light in a plurality of wavelengths. When a plurality of wavelengths are used, the oxygen saturation can be calculated as functional information. For example, photoacoustic signals may be acquired by switching the two wavelengths alternately in each imaging cycle, the reconstructed image data may be calculated therefrom, and the oxygen saturation may be calculated on the basis of the calculated reconstructed image data. A method of calculating the oxygen saturation is well known, and therefore detailed description thereof has been omitted.

Furthermore, the plurality of embodiments described above may be packaged in a single photoacoustic device so that it is possible to switch therebetween. Moreover, a function for transmitting an ultrasonic wave from the transducer and a function for receiving an ultrasonic echo reflected by the subject and implementing measurement on the basis of the ultrasonic echo may be added to the photoacoustic device according to the present invention.

Embodiment(s) of the present invention can also be realized by a computer of a system or apparatus that reads out and executes computer executable instructions (e.g., one or more programs) recorded on a storage medium (which may also be referred to more fully as ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-96747, filed on May 15, 2017, which is hereby incorporated by reference herein in its entirety.

What is claimed is:

1. A subject information acquisition device comprising:
a light source for emitting light to a subject;
an acoustic wave detection unit configured to receive an acoustic wave generated by the subject in response to the light, and convert the received acoustic wave into an electric signal;
a signal processing unit configured to implement emission of the light and acquisition of the electric signal at a non-periodic sampling timing, and add together electric signals acquired in time series at each sampling timing; and
an image generation unit configured to generate an image representing characteristic information of the subject on the basis of the added electric signals.

2. The subject information acquisition device according to claim 1,
wherein the image generation unit generates the image at an imaging cycle corresponding to a predetermined frame rate, and
wherein an interval between adjacent sampling timings is shorter than the imaging cycle.

3. The subject information acquisition device according to claim 1, further comprising a display unit configured to display the image at a display cycle corresponding to a predetermined frame rate,
wherein an interval between adjacent sampling timings is shorter than the display cycle.

4. The subject information acquisition device according to claim 1, wherein the signal processing unit selects a pattern to be used from a plurality of patterns defining the sampling timing.

5. The subject information acquisition device according to claim 1, wherein the sampling timing varies randomly.

6. The subject information acquisition device according to claim 1, wherein an interval between sampling timings increases or decreases monotonically at each sampling timing.

7. The subject information acquisition device according to claim 1, wherein the acoustic wave detection unit comprises:
a plurality of acoustic wave detection elements; and
an A/D conversion unit configured to convert reception signals, generated when the plurality of acoustic wave detection elements each receive the acoustic wave, into digital signals, and output each of the digital signals as the electric signal.

8. A subject information acquisition method comprising:
an emission step for emitting light;
an acoustic wave detection step for receiving an acoustic wave generated by the subject in response to the light, and converting the received acoustic wave into an electric signal;
a signal processing step for implementing emission of the light and acquisition of the electric signal at a non-periodic sampling timing, and adding together electric signals acquired in time series at each sampling timing; and
an image generation step for generating an image representing characteristic information of the subject on the basis of the added electric signals.

9. The subject information acquisition method according to claim 8, wherein, in the image generation step, the image is generated at an imaging cycle corresponding to a predetermined frame rate, and
wherein an interval between adjacent sampling timings is shorter than the imaging cycle.

10. The subject information acquisition method according to claim 8, further comprising a display step for displaying the image at a display cycle corresponding to a predetermined frame rate,
wherein an interval between adjacent sampling timings is shorter than the display cycle.

11. The subject information acquisition method according to claim 8, wherein, in the signal processing step, a pattern to be used is selected from a plurality of patterns defining the sampling timing.

12. A non-transitory computer-readable storage medium storing a computer program which, when run by a computer, causes the computer to execute each step of the method according to claim 8.
